(19) United States
(12) Patent Application Publication
(10) Pub. No.: US 2006/0019995 A1
(43) Pub. Date: Jan. 26, 2006

(54) 2,3-dihydro-4(1H)-pyridone derivatives , method for production thereof and pharmaceutical composition comprising the same

(76) Inventors: Sylvain Rault, Moult (FR); Nicolas Leflemme, Bissieres (FR); Patrick Dallemagne, Saint Georges d' Aunay (FR); Pierre Lestage, La Cell Saint Cloud (FR); Brian Lockhart, Feucherolles (FR); Laurence Danober, Montesson (FR); Bruno Pfeiffer, Saint Leu La Foret (FR); Pierre Renard, Le Chesnay (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 10/533,784

(22) PCT Filed: Nov. 4, 2003
(86) PCT No.: PCT/FR03/03276

§ 371 Date: May 4, 2005

(30) Foreign Application Priority Data
Nov. 5, 2002 (FR) 02/13803

(51) Int. Cl. A61K 31/444; A61K 31/4436; C07D 409/02; C07D 401/02; C07D 211/72

(52) U.S. Cl. 514/332; 546/255; 546/290; 546/280.4; 514/342; 514/345

(57) ABSTRACT

A compound selected from those of formula (I): 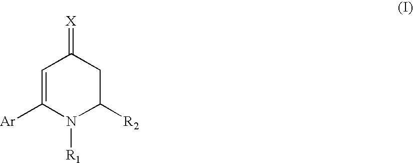 wherein:
R₁ represents hydrogen, aryl(C₁-C₆)alkyl, linear or branched (C₁-C₆)alkyl, linear or branched (C₁-C₆)acyl, linear or branched (C₁-C₆)alkoxycarbonyl, aryl(C₁-C₆)alkoxycarbonyl, or trifluoroacetyl,
R₂ represents hydrogen, linear or branched (C₁-C₆)alkyl,
X represents oxygen or NOR₃,
R₃ represents hydrogen, linear or branched (C₁-C₆)alkyl optionally substituted by one or more identical or different groups selected from hydroxy, amino and linear or branched (C₁-C₆)alkoxy, Ar represents aryl or heteroaryl, its isomers, and addition salts thereof with a pharmaceutically acceptable acid or base, and medicinal products containing the same which are useful in the treatment of cognitive disorders and which possess antalgic properties.

The present invention relates to new 2,3-dihydro-4(1H)-pyridone compounds, to a process for their preparation, to pharmaceutical compositions containing them and to their use as facilitators of memory and cognition and as antalgic agents.

Ageing of the population due to increased life expectancy has brought with it a major increase in cognitive disorders associated with normal cerebral ageing and with pathological cerebral ageing occurring in the course of neurodegenerative diseases such as, for example, Alzheimer's disease.

The majority of substances used today in treating cognitive disorders associated with ageing act by facilitating the central cholinergic systems—either directly, as in the case of acetylcholinesterase inhibitors (tacrine, donepezil) and cholinergic agonists (nefiracetam), or indirectly, as in the case of nootropic agents (piracetam, pramiracetam) and cerebral vasodilators (vinpocetine).

Besides their cognitive properties, substances acting directly on the central cholinergic systems often have antalgic properties but also have hypothermic properties, which can be undesirable.

It has therefore been especially valuable to synthesise new compounds that are capable of opposing the cognitive disorders associated with ageing and/or of improving cognitive processes and that can possess antalgic properties without having hypothermic activity.

4-Hydroxy- or 4-oxo-substituted 1-aza-2-alkyl-6-aryl-cycloalkanes and 1-aza-2-alkyl-6-aryl-cycloalkenes have already been described in the literature (J. Org. Chem. 1988, 53, 2426; Liebigs Ann. Chem. 1986, 11, 1823; Synlett 1993, 9, 657; Tet. Lett. 1998, 39(3/4), 217), but no pharmacological activity has been described for those compounds. Patent application EP 0119087 describes 1-aza-2-alkyl-6-aryl-cycloalkane compounds for use as antalgic agents.

More specifically, the present invention relates to compounds of formula (I): 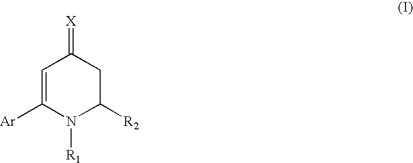
wherein:
R₁ represents a hydrogen atom or an aryl(C₁-C₆)alkyl group in which the alkyl moiety may be linear or branched, a linear or branched (C₁-C₆)alkyl group, a linear or branched (C₁-C₆)acyl group, a linear or branched (C₁-C₆)alkoxycarbonyl group, an aryl(C₁-C₆)— alkoxycarbonyl group in which the alkoxy moiety may be linear or branched, or a trifluoroacetyl group,
R₂ represents a linear or branched (C₁-C₆)alkyl group,
X represents an oxygen atom or NOR₃ wherein:
   R₃ represents a hydrogen atom or a linear or branched (C₁-C₆)alkyl group optionally substituted by one or more identical or different groups selected from hydroxy, amino (optionally substituted by one or two linear or branched (C₁-C₆)-alkyl groups) and linear or branched (C₁-C₆)alkoxy,
Ar represents an aryl group or a heteroaryl group,
   to their enantiomers, diastereoisomers and also to addition salts thereof with a pharmaceutically acceptable acid, it being understood that aryl is understood to be a phenyl, biphenyl, naphthyl or tetrahydronaphthyl group, each of those groups being optionally substituted by one or more identical or different groups selected from halogen, linear or branched (C₁-C₆)alkyl, hydroxy, linear or branched (C₁-C₆)alkoxy, trihalomethyl, nitro and amino (optionally substituted by one or more linear or branched (C₁-C₆)alkyl groups),
   and a heteroaryl group is understood to be an aromatic, mono- or bi-cyclic, 5- to 12-membered group containing one, two or three hetero atoms selected from oxygen, nitrogen and sulphur, it being understood that the heteroaryl group may be optionally substituted by one or more identical or different groups selected from halogen, linear or branched (C₁-C₆)alkyl, hydroxy, linear or branched (C₁-C₆)alkoxy, trihalomethyl, nitro and amino (optionally substituted by one or more linear or branched (C₁-C₆)alkyl groups). Among the heteroaryl groups there may be mentioned, without implying any limitation, thienyl, pyridyl, furyl, pyrrolyl, imidazolyl, oxazolyl, isoxazolyl, thiazolyl and isothiazolyl groups.

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, benzenesulphonic acid, camphoric acid etc.

The preferred compounds of formula (I) are those wherein the group X represents an oxygen atom.

The group R₁ to which preference is given in accordance with the invention is a hydrogen atom or a linear or branched (C₁-C₆)alkoxycarbonyl group.

The term aryl used in respect of the group Ar as defined for formula (I) is preferably an optionally substituted phenyl group.

The term aryl used in respect of the group Ar as defined for formula (I) is more preferably a substituted phenyl group.

The term heteroaryl used in respect of the group Ar as defined for formula (I) is preferably an optionally substituted thienyl group or an optionally substituted pyridyl group.

The invention relates more especially to the compounds of formula (I) which are:
tert-butyl 2-methyl-4-oxo-6-(2-thienyl)-3,4-dihydro-1 (2H)-pyridinecarboxylate
2-methyl-6-(2-thienyl)-2,3-dihydro-4(1H)-pyridone
tert-butyl 2-methyl-4-oxo-6-phenyl-3,4-dihydro-1 (2H)-pyridinecarboxylate
2-methyl-6-phenyl-2,3-dihydro-4(1H)-pyridone
tert-butyl 6-(3-chlorophenyl)-2-methyl-4-oxo-3,4-dihydro-1 (2H)-pyridine-carboxylate
6-(3-chlorophenyl)-2-methyl-2,3-dihydro-4(1H)-pyridone
tert-butyl 6-(6-chloro-3-pyridyl)-2-methyl-4-oxo-3,4-dihydro-1(2H)-pyridine-carboxylate
6-(6-chloro-3-pyridyl)-2-methyl-2,3-dihydro-4(1H)-pyridone.

The enantiomers, diastereoisomers and also the addition salts with a pharmaceutically acceptable acid of the preferred compounds form an integral part of the invention.

The invention relates also to a process for the preparation of compounds of formula (I), characterised in that 4-methoxypyridine is reacted in succession with phenyl chloroformate, with an organomagnesium compound of formula (II):
R₂MgBr (II)
wherein R₂ is as defined for formula (I),
and with potassium tert-butoxide to yield a compound of formula (III): 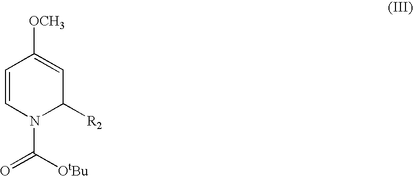
   wherein R₂ is as defined hereinbefore,
   which compound of formula (III) is reacted with butyllithium and with iodine to yield an iodated compound of formula (IV): 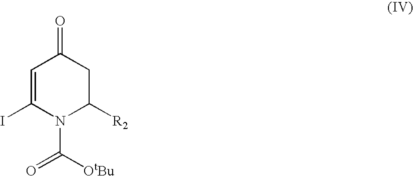
   wherein R₂ is as defined hereinbefore,
   which compound of formula (IV) is reacted, in the presence of tetrakis(triphenylphosphine)palladium(0), with a boronic acid of formula (V):
   ArB(OH)₂ (V)
   wherein Ar is as defined for formula (I),
   to yield a compound of formula (I/a), which is a particular case of the compounds of formula (I): 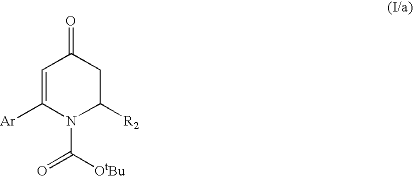
   wherein Ar and R₂ are as defined hereinbefore,
   in which compound of formula (I/a) the amine function is optionally deprotected according to conventional techniques of organic synthesis to yield a compound of formula (I/b), which is a particular case of the compounds of formula (I): 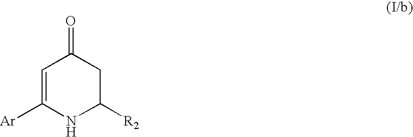
   wherein R₂ and Ar are as defined hereinbefore,
   which compound of formula (IIb) is optionally reacted with a compound of the formula R′₁Y wherein R′₁ represents an aryl(C₁-C₆)alkyl group in which the alkyl moiety may be linear or branched, a linear or branched (C₁-C₆)alkyl group, a linear or branched (C₁-C₆)acyl group, a linear or branched (C₁-C₆)alkoxycarbonyl group, an aryl(C₁-C₆)— alkoxycarbonyl group in which the alkoxy moiety may be linear or branched, or a trifluoroacetyl group, and Y represents a leaving group, to yield a compound of formula (I/c), which is a particular case of the compounds of formula (I): 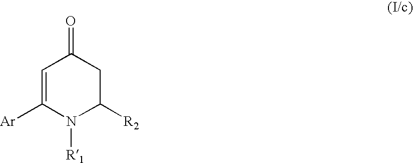
   wherein Ar, R′₁ and R₂ are as defined hereinbefore,
   the compounds of formulae (I/b) and (I/c) forming the compounds of formula (I/d): 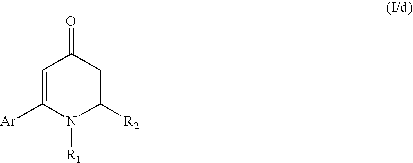
   wherein Ar, R₁ and R₂ are as defined hereinbefore,
   which compounds of formula (I/d) are optionally reacted with a compound of the formula H₂N—OR₃ wherein R₃ is as defined for formula (I), to yield a compound of formula (I/e), which is a particular case of the compounds of formula (I): 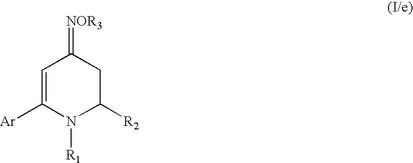
   wherein Ar, R₁, R₂ and R₃ are as defined hereinbefore,
   the compounds of formulae (I/a) to (I/e) constituting the totality of the compounds of formula (I), which are purified, where necessary, according to conventional purification techniques, are separated, if desired, into their isomers according to conventional separation techniques and are converted, if desired, into their addition salts with a pharmaceutically acceptable acid.

In addition to the fact that the compounds of the present invention are new, they exhibit properties facilitating cognitive processes and antalgic properties, rendering them of use in the treatment of cognitive deficiencies associated with cerebral ageing and with neurodegenerative pathologies, such as Alzheimer's disease, Parkinson's disease, Pick's disease, Korsakoffs disease and frontal lobe and subcortical dementias and in the treatment of pain.

The invention relates also to pharmaceutical compositions comprising as active ingredient a compound of formula (I) together with one or more appropriate, inert, non-toxic excipients. Among the pharmaceutical compositions according to the invention there may be mentioned more especially those that are suitable for oral, parenteral (intravenous or subcutaneous) and nasal administration, tablets or dragees, sublingual tablets, gelatin capsules, lozenges, suppositories, creams, ointments, dermal gels, injectable preparations, drinkable suspensions etc.

The dosage used can be adapted according to the nature and the severity of the disorder, the administration route and the age and weight of the patient. The dosage varies from 1 to to 500 mg per day in one or more administrations.

The following Examples illustrate the invention without limiting it in any way.

The starting materials used are products that are known or prepared according to known procedures.

The structures of the compounds described in the Examples were determined according to customary spectrophotometric techniques (infra-red, nuclear magnetic resonance, mass spectrometry).

### PREPARATION 1: Tert-butyl 4-methoxy-2-methyl-1(2H)-pyridinecarboxylate

37.81 mmol of ethyl chloroformate are added to a solution, cooled to −25° C., of 37.43 mmol of 4-methoxypyridine in 100 ml of anhydrous tetrahydrofuran under an argon atmosphere. After one hour's stirring at −25° C., 39.30 mmol of 3M methylmagnesium bromide are added dropwise. The reaction mixture is stirred for 30 minutes at −25° C. and then for one hour at ambient temperature. 100 ml of water are then added and the aqueous phase is then extracted twice with diethyl ether, dried over magnesium sulphate, filtered and then concentrated under reduced pressure. The resulting oil is taken up in 100 ml of anhydrous tetrahydrofuran, the solution is then cooled to −40° C., and then 0.15 mmol of potassium tert-butoxide is added. The reaction mixture is stirred for 2 hours at −40° C. and for one hour at ambient temperature, and 100 ml of water are then added. The aqueous phase is extracted twice with diethyl ether and then the organic phase is dried over magnesium sulphate, filtered and concentrated under reduced pressure to give the expected product.

### PREPARATION 2: Tert-butyl 6-iodo-2-methyl-4-oxo-3,4-dihydro-1(2H)-pyridine-carboxylate

40.48 mmol of n-butyllithium are added to a solution, at −60° C., of 33.73 mmol of the compound of Preparation 1 in 100 ml of anhydrous tetrahydrofuran under an argon atmosphere. Stirring is carried out for 30 minutes at −60° C., and then 37.11 mmol of iodine are added. After stirring for 2 hours at −60° C. and then for one hour at ambient temperature, 100 ml of a 1N aqueous hydrochloric acid solution are added to the reaction mixture. The aqueous phase is extracted twice with diethyl ether, and the organic phase is dried over magnesium sulphate, filtered and concentrated under reduced pressure. Purification by chromatography on silica gel (diethyl ether/petroleum ether: 4/6) yields the expected product.

IR(KBr): v_{C═O}=1668, 1722 cm⁻¹.

### EXAMPLE 1

### Tert-butyl 2-methyl-4-oxo-6-(2-thienyl)-3,4-dihydro-1(2H)-pyridinecarboxylate

There are introduced into a 100 ml flask 4.45 mmol of the compound of Preparation 2, 0.22 mmol of tetrakis(triphenylphosphine)palladium(0) and 20 ml of dimethoxyethane, then 5.34 mmol of thiophene-2-boronic acid and 11.12 mmol of sodium hydrogen carbonate dissolved in 20 ml of water. The reaction mixture is heated under reflux and with vigorous stirring for about 5 hours. After cooling, the aqueous phase is extracted twice with chloroform and the organic phase is dried over calcium chloride, filtered and concentrated under reduced pressure. Purification by chromatography on silica gel (diethyl ether/petroleum ether: 4/6) yields the expected product.

Melting point: 90° C.
IR (KBr): v_{C - O} = 1659, 1718 cm⁻¹.
Elemental microanalysis:

| | % C | % H | % N |
|---|---|---|---|
| calculated | 61.41 | 6.53 | 4.77 |
| found | 61.34 | 6.71 | 4.86 |

### EXAMPLE 2

### 2-Methyl-6-(2-thienyl)-2,3-dihydro-4(1H)-pyridone

2.73 mmol of the compound of Example 1, 10 ml of dichloromethane and 27.27 mmol of trifluoroacetic acid are mixed. The reaction mixture is stirred at ambient temperature for 4 hours and then rendered alkaline by the addition of a saturated aqueous potassium carbonate solution. The aqueous phase is extracted twice with dichloromethane, and the organic phases are combined and then dried over calcium chloride, filtered and concentrated under reduced pressure. Purification by chromatography on silica gel (ethyl acetate) yields the expected product.

Melting point: 155° C.
IR (KBr): v_{C = O} = 1605 cm⁻¹; v_{NH} = 3288 cm⁻¹.
Elemental microanalysis:

| | % C | % H | % N |
|---|---|---|---|
| calculated | 62.15 | 5.74 | 7.24 |
| found | 62.34 | 5.62 | 7.02 |

### EXAMPLE 3

### Tert-butyl 2-methyl-4-oxo-6-phenyl-3,4-dihydro-1(2H)-pyridine-carboxylate

The expected product is obtained according to the process described in Example 1, using phenylboronic acid.

Melting point: 99° C.
IR (KBr): v_{C = O} = 1655, 1709 cm⁻¹.
Elemental microanalysis:

| | % C | % H | % N |
|---|---|---|---|
| calculated | 71.06 | 7.37 | 4.87 |
| found | 70.92 | 7.51 | 4.71 |

### EXAMPLE 4

### 2-Methyl-6-phenyl-2,3-dihydro-4(1H)-pyridone

The expected product is obtained according to the process described in Example 2, starting from the compound of Example 3.

Melting point: 161° C.
IR (KBr): v_{C = O} = 1605 cm⁻¹; v_{NH} = 3268 cm⁻¹.
Elemental microanalysis.

| | % C | % H | % N |
|---|---|---|---|
| calculated | 76.98 | 7.00 | 7.48 |
| found | 77.21 | 7.06 | 7.22 |

### EXAMPLE 5

### Tert-butyl 6-(3-chlorophenyl)-2-methyl-4-oxo-3,4-dihydro-1(2H)-pyridinecarboxylate

The expected product is obtained according to the process described in Example 1, using 3-chlorobenzeneboronic acid.

Melting point: 101° C.
IR (KBr): v_{C = O} = 1674, 1714 cm⁻¹.
Elemental microanalysis:

| | % C | % H | % N |
|---|---|---|---|
| calculated | 63.45 | 6.26 | 4.35 |
| found | 63.39 | 6.36 | 4.21 |

### EXAMPLE 6

### 6-(3-Chlorophenyl)-2-methyl-2,3-dihydro-4(1H)-pyridone

The expected product is obtained according to the process described in Example 2, starting from the compound of Example 5.

Melting point: 133° C.
IR (KBr): v_{C = O} = 1605 cm⁻¹; v_{NH} = 3255 cm⁻¹.
Elemental microanalysis:

| | % C | % H | % N |
|---|---|---|---|
| calculated | 65.02 | 5.46 | 6.32 |
| found | 65.15 | 5.59 | 6.13 |

### EXAMPLE 7

### Tert-butyl 2-methyl-4-oxo-6-(6-chloro-3-pyridyl)-3,4-dihydro-1(2H)-pyridinecarboxylate

The expected product is obtained according to the process described in Example 1, using 6-chloropyridine-3-boronic acid.

Melting point: 115° C.
IR (KBr): v_{C = O} = 1660, 1711 cm⁻¹.
Elemental microanalysis:

| | % C | % H | % N |
|---|---|---|---|
| calculated | 59.54 | 5.93 | 8.68 |
| found | 59.75 | 5.88 | 8.42 |

### EXAMPLE 8

### 6-(6-Chloro-3-pyridyl)-2-methyl-2,3-dihydro-4(1H)-pyridone

The expected product is obtained according to the process described in Example 2, starting from the compound of Example 7.

Melting point: 216° C.
IR (KBr): v_{C = O} = 1613 cm⁻¹; v_{NH} = 3256 cm⁻¹.
Elemental microanalysis:

| | % C | % H | % N |
|---|---|---|---|
| calculated | 59.33 | 4.98 | 12.58 |
| found | 59.19 | 5.08 | 12.39 |

Pharmacological Study of Compounds of the Invention

### EXAMPLE 9

### Body Temperature in the NMRI Mouse

The effects of the compounds of the present invention on body temperature were assessed in the adult male NMRI mouse. The rectal temperature of the mice (18-20 g) was measured just before pharmacological treatment (intraperitoneal route) with the compounds being studied or their carriers (20 mg/kg). The mice were then placed in individual cages (10×10×10 cm) and their rectal temperature was measured every 30 minutes during the 2 hours following treatment. The values were the means (° C.) plus or minus the standard errors of the means, and inter-group comparisons were carried out by a single-factor variance analysis test followed, where appropriate, by a Dunnett test.

The results show that the compounds of the invention do not have hypothermic activity at doses up to 20 mg/kg.

### EXAMPLE 10

### Abdominal Contractions Induced by phenyl-p-benzoquinone (PBQ) in the NMRI Mouse

Intraperitoneal administration of an alcoholic solution of PBQ causes abdominal cramps in the mouse (SIEGMUND et al., Proc. Soc. Exp. Biol., 1957, 95, 729-731). The cramps are characterised by repeated contractions of the abdominal musculature, accompanied by extension of the hind limbs. Most analgesics antagonise these abdominal cramps (COLLIER et al., Brit. J. Pharmacol. Chem., 1968, 32, 295-310). At t=0 min., the animals are weighed and the compound being studied is administered by the IP route. A group of control animals is given the solvent used for the compound. At t=30 min., an alcoholic solution of PBQ (0.2%) is administered by the IP route in a volume of 0.25 ml/mouse. Immediately after administration of the PBQ, the animals are placed in cylinders of plexiglass (L=19.5 cm; I.D.=5 cm). From t=35 min. to t=45 min., the animals' reaction is observed and the experimenter notes the total number of abdominal cramps per animal. The table below shows the percentage inhibition of the number of abdominal cramps measured in the control animals, at the active dose of the compound studied.

The results obtained show that the compounds of the invention possess antalgic properties.

| Example | Dose (mg/kg) | Inhibition (%) |
|---|---|---|
| 2 | 20 | 48% |
| 3 | 20 | 59% |
| 6 | 20 | 48% |

### EXAMPLE 11

### Social Recognition in the Wistar Rat

Initially described in 1982 by THOR and HOLLOWAY (J. Comp. Physiol., 1982, 96, 1000-1006), the social recognition test has subsequently been proposed by various authors (DANTZER et al., Psychopharmacology, 1987, 91, 363-368; PER10 et al., Psycho-pharmacology, 1989, 97, 262-268) for studying the mnemocognitive effects of new compounds. The test is based on the natural expression of the olfactory memory of the rat and its natural tendency to forget and allows evaluation of memorisation, by recognition of a young congeneric animal, by an adult rat. A young rat (21 days), taken at random, is placed for 5 minutes in the cage housing an adult rat. With the aid of a video device, the experimenter observes the social recognition behaviour of the adult rat and measures its overall duration. The young rat is then removed from the adult rat's cage and is placed in its own cage until the second introduction. The adult rat is then given the compound under test (intraperitoneal route) and, after 2 hours, is again brought into the presence (5 minutes) of the young rat. The social recognition behaviour is then observed again and its duration measured. The table below shows the difference (T₂-T₁), expressed in seconds, between the “recognition” times of the 2 encounters.

The results obtained show that the compounds of the invention very greatly enhance memorisation, even at a low dose.

| Example | Dose (mg/kg) | T₂ − T₁ (s) ± sem |
|---|---|---|
| 6 | 3 | −21.4 ± 5.1 |
| 3 | 3 | −25.3 ± 7.1 |
| 1 | 3 | −17.4 ± 2.5 |
| 8 | 3 | −17.2 ± 4.6 |

### EXAMPLE 12

### Pharmaceutical Composition

Formulation for the preparation of 1000 tablets each comprising 10 mg of active ingredient:

| | |
|---|---|
| Compound of Example 1 | 10 g |
| Hydroxypropylcellulose | 2 g |
| Wheat starch | 10 g |
| Lactose | 100 g |
| Magnesium stearate | 3 g |
| Talc | 3 g |

1-9. (canceled)

10. A compound of formula (I): 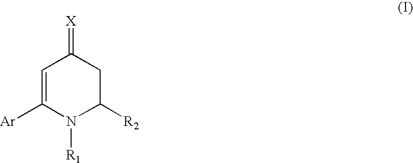
wherein:
R₁ represents hydrogen, aryl(C₁-C₆)alkyl in which the alkyl moiety may be linear or branched, linear or branched (C₁-C₆)alkyl, linear or branched (C₁-C₆)acyl, linear or branched (C₁-C₆)alkoxycarbonyl, aryl(C₁-C₆)alkoxycarbonyl in which the alkoxy moiety may be linear or branched, or trifluoroacetyl,
R₂ represents linear or branched (C₁-C₆)alkyl,
X represents oxygen or NOR₃ wherein:
R₃ represents hydrogen, linear or branched (C₁-C₆)alkyl optionally substituted by one or more identical or different groups selected from hydroxy, amino (optionally substituted by one or two linear or branched (C₁-C₆)alkyl groups) and linear or branched (C₁-C₆)alkoxy,
Ar represents aryl or heteroaryl,
it being understood that:
aryl is a phenyl, biphenyl, naphthyl or tetrahydronaphthyl group, each of those groups being optionally substituted by one or more identical or different substituents selected from halogen, linear or branched (C₁-C₆)alkyl, hydroxy, linear or branched (C₁-C₆)alkoxy, trihalomethyl, nitro and amino (optionally substituted by one or more linear or branched (C₁-C₆)alkyl groups),
and heteroaryl is an aromatic, monocyclic or bicyclic heterocycle that has from 5 to 12 ring atoms, and which optionally contains in the ring system, two or three hetero atoms selected from oxygen, nitrogen and sulphur, it being understood that heteroaryl may be optionally substituted by one or more identical or different substituents selected from halogen, linear or branched (C₁-C₆)alkyl, hydroxy, linear or branched (C₁-C₆)alkoxy, trihalomethyl, nitro and amino (optionally substituted by one or more linear or branched (C₁-C₆)alkyl groups).

11. A compound of claim 10, wherein X represents oxygen.

12. A compound of claim 10, wherein R₁ represents hydrogen or linear or branched (C₁-C₆) alkoxycarbonyl.

13. A compound of claim 10, wherein Ar represents an optionally substituted phenyl.

14. A compound of claim 10, wherein Ar represents a substituted phenyl.

15. A compound of claim 10, wherein Ar represents an optionally substituted thienyl or an optionally substituted pyridyl.

16. A compound of claim 10 which is selected from:
tert-butyl 2-methyl-4-oxo-6-(2-thienyl)-3,4-dihydro-1 (2H)-pyridinecarboxylate,
2-methyl-6-(2-thienyl)-2,3-dihydro-4(1H)-pyridone,
tert-butyl 2-methyl-4-oxo-6-phenyl-3,4-dihydro-1 (2H)-pyridinecarboxylate,
2-methyl-6-phenyl-2,3-dihydro-4(1H)-pyridone,
tert-butyl 6-(3-chlorophenyl)-2-methyl-4-oxo-3,4-dihydro-1 (2H)-pyridine-carboxylate,
6-(3-chlorophenyl)-2-methyl-2,3-dihydro-4(1H)-pyridone,
tert-butyl 6-(6-chloro-3-pyridyl)-2-methyl-4-oxo-3,4-dihydro-1 (2H)-pyridine-carboxylate and
6-(6-chloro-3-pyridyl)-2-methyl-2,3-dihydro-4(1H)-pyridone.

17. A method for treating a living animal body, including a human, afflicted with a disorder of memory and cognition comprising the step of administering to the living animal body, including a human, an amount of a compound of claim 10, which is effective for alleviation of the disorder.

18. A pharmaceutical composition useful in treating memory and cognition disorders comprising as active principle an effective amount of a compound of claim 10, together with are as more pharmaceutically acceptable excipients or vehicles.
